(19) United States
(12) Patent Application Publication
(10) Pub. No.: US 2004/0220551 A1
(43) Pub. Date: Nov. 4, 2004

(54) Low profile components for patient infusion device

(76) Inventors: J. Christopher Flaherty, Topsfield, MA (US); William Gorman, South Hamilton, MA (US); Steven Diianni, Danvers, MA (US)

Correspondence Address: INSULET CORPORATION, 100 CUMMINGS CENTER, SUITE 239G, BEVERLY, MA, 01915, US

(21) Appl. No.: 10/426,494

(22) Filed: Apr. 30, 2003

(51) Int. Cl. A61K 9/22

(52) U.S. Cl. 604/890.1; 700/282; 128/903; 128/904; 340/825.37

(57) ABSTRACT

A device for delivering fluid to a patient, including a reservoir, a dispenser for causing fluid to flow from the reservoir, a local processor connected to the dispenser and programmed to cause a flow of fluid from the reservoir based solely on flow instructions from a separate, remote control device, a power supply connected to the local processor, a wireless receiver connected to the local processor for receiving the flow instructions from a separate, remote control device and delivering the flow instructions to the local processor, and a housing containing the reservoir, the dispenser, the local processor, the power supply and the wireless receiver. At least two of the reservoir, the dispenser and the power supply are vertically stacked within the housing and at least one of the dispenser and the power supply has a horizontal cross-sectional area that is greater than fifty percent of a cross-sectional area of the housing.

### CROSS-REFERENCE TO RELATED APPLICATIONS

The present application is related to co-pending U.S. patent application Ser. No. 09/943,992, filed on Aug. 31, 2001 (Atty. Docket No. INSL-110), and entitled DEVICES, SYSTEMS AND METHODS FOR PATIENT INFUSION, which is assigned to the assignee of the present application and incorporated herein by reference.

### FIELD OF THE INVENTION

The present invention relates generally to medical devices, systems and methods, and more particularly to small, low cost, portable infusion devices and methods that are useable to achieve precise, sophisticated, and programmable flow patterns for the delivery of therapeutic liquids such as insulin to a mammalian patient. Even more particularly, the present invention is directed to various new and improved low profile components for an infusion device.

### BACKGROUND OF THE INVENTION

Ambulatory infusion pumps have been developed for delivering liquid medicaments to a patient. These infusion devices have the ability to offer sophisticated fluid delivery profiles accomplishing bolus requirements, continuous infusion and variable flow rate delivery. These infusion capabilities usually result in better efficacy of the drug and therapy and less toxicity to the patient's system. An example of a use of an ambulatory infusion pump is for the delivery of insulin for the treatment of diabetes mellitus. These pumps can deliver insulin on a continuous basal basis as well as a bolus basis as is disclosed in U.S. Pat. No. 4,498,843 to Schneider et al.

Currently available ambulatory infusion devices are expensive, difficult to program and prepare for infusion, and tend to be bulky, heavy and very fragile. Filling these devices can be difficult and require the patient to carry both the intended medication as well as filling accessories. The devices require specialized care, maintenance, and cleaning to assure proper functionality and safety for their intended long term use. Due to the high cost of existing devices, healthcare providers limit the patient populations approved to use the devices and therapies for which the devices can be used.

Clearly, therefore, there was a need for a programmable and adjustable infusion system that is precise and reliable and can offer clinicians and patients a small, low cost, light-weight, easy-to-use alternative for parenteral delivery of liquid medicines.

In response, the applicant of the present application provided a small, low cost, light-weight, easy-to-use device for delivering liquid medicines to a patient. The device, which is described in detail in co-pending U.S. application Ser. No. 09/943,992, filed on Aug. 31, 2001, includes an exit port, a dispenser for causing fluid from a reservoir to flow to the exit port, a local processor programmed to cause a flow of fluid to the exit port based on flow instructions from a separate, remote control device, and a wireless receiver connected to the local processor for receiving the flow instructions. To reduce the size, complexity and costs of the device, the device is provided with a housing that is free of user input components, such as a keypad, for providing flow instructions to the local processor.

What is still desired, however, are new and improved components, such as motors for example, for devices for delivering liquid medicines to a patient. Preferably, the components will have relatively low profiles (i.e., heights) so that the resulting fluid delivery device also has a low profile when attached to the skin of a patient. A low profile fluid delivery device is desirable since a low profile device is less discrete during use, can more easily fit under the clothing of a patient when attached to the patient's skin, and a low profile fluid delivery device is less likely to be accidentally removed from the patient's skin.

### SUMMARY OF THE INVENTION

The present invention provides a device for delivering fluid to a patient, including a reservoir, a dispenser for causing fluid to flow from the reservoir, a local processor connected to the dispenser and programmed to cause a flow of fluid from the reservoir based solely on flow instructions from a separate, remote control device, a power supply connected to the local processor, a wireless receiver connected to the local processor for receiving the flow instructions from a separate, remote control device and delivering the flow instructions to the local processor, and a housing containing the reservoir, the dispenser, the local processor, the power supply and the wireless receiver. At least two of the reservoir, the dispenser and the power supply are vertically stacked within the housing and at least one of the dispenser and the power supply has a horizontal cross-sectional area that is greater than fifty percent of a cross-sectional area of the housing.

The components of the fluid delivery device of the present invention have relatively low profiles (i.e., heights) so that the resulting fluid delivery device also has a relatively low profile when attached to the skin of a patient. Among other features and benefits, the low profile fluid delivery device is less discrete during use, can more easily fit under the clothing of a patient when attached to the patient's skin, and is less likely to be accidentally removed from the patient's skin. Moreover, the low profile nature and vertical assembly of the components of the fluid delivery device lends the device to mass production techniques so that devices constructed in accordance with the present invention can be made relatively cheaply and can be disposable in nature.

These aspects of the invention together with additional features and advantages thereof may best be understood by reference to the following detailed descriptions and examples taken in connection with the accompanying illustrated drawings.

### BRIEF DESCRIPTION OF THE DRAWINGS

FIG. 1 is a perspective view of an exemplary embodiment of a fluid delivery device constructed in accordance with the present invention shown secured on a patient, and a remote control device for use with the fluid delivery device (the remote control device being enlarged with respect to the patient and the fluid delivery device for purposes of illustration);

FIG. 2 is a schematic side and top perspective view illustrating internal components of the fluid delivery device of FIG. 1;

FIG. 3 is a schematic top plan view illustrating the internal components of the fluid delivery device of FIG. 1;

FIG. 4 is a schematic, exploded side and top perspective view of the fluid delivery device of FIG. 1;

FIG. 5 is a schematic side view of another exemplary embodiment of a fluid delivery device constructed in accordance with the present invention;

FIG. 6 is a schematic top view of the fluid delivery device of FIG. 5;

FIG. 7 is an exploded side and top perspective view of components of the fluid delivery device of FIG. 5;

FIG. 8 is a schematic side view of an additional exemplary embodiment of a fluid delivery device constructed in accordance with the present invention;

FIG. 9 is a schematic top view of the fluid delivery device of FIG. 8;

FIGS. 10 and 11 are schematic top views illustrating operation of an exemplary embodiment of a component of a fluid delivery device constructed in accordance with the present invention;

FIGS. 12 and 13 are schematic top views illustrating operation of another exemplary embodiment of a component of a fluid delivery device constructed in accordance with the present invention;

FIG. 14 is a schematic top view of an additional exemplary embodiment of a component of a fluid delivery device constructed in accordance with the present invention;

FIG. 15 is a schematic side view of the fluid delivery device of FIG. 14;

FIG. 16 is a schematic side view of a further exemplary embodiment of a fluid delivery device constructed in accordance with the present invention;

FIG. 17 is a schematic top view of an upper component of the fluid delivery device of FIG. 16;

FIG. 18 is a schematic top view of a lower component of the fluid delivery device of FIG. 16;

FIG. 19 is a sectional side view of another exemplary embodiment of a fluid delivery device constructed in accordance with the present invention, including a flexible fluid reservoir sandwiched between a rigid backing plate and a spiral pin guide;

FIG. 20 is a top sectional view of the fluid delivery device of FIG. 19, showing the pin guide removed from the reservoir;

FIG. 21 is a top plan view of the reservoir of the fluid delivery device of FIG. 19;

FIG. 22 is an exploded side elevation view of the reservoir of the fluid delivery device of FIG. 19;

FIG. 23 is a sectional side view of another exemplary embodiment of a fluid delivery device constructed in accordance with the present invention, including a gear train constructed in accordance with the present invention;

FIG. 24 is a top sectional view of the fluid delivery device of FIG. 23, showing the gear train;

FIG. 25 is a top plan view of an exemplary embodiment of a motor constructed in accordance with the present invention for use with a fluid delivery device;

FIG. 26 is a sectional view of the motor taken along line 26-26 of FIG. 25;

FIG. 27 is a top plan view of another exemplary embodiment of a motor constructed in accordance with the present invention for use with a fluid delivery device; and

FIG. 28 is a sectional view of the motor taken along line 28-28 of FIG. 27.

It should be noted that components shown in the drawings are not made to scale and are not necessarily shown in actual proportion to one another. Like reference characters designate identical or corresponding components and units throughout the several views.

### DETAILED DESCRIPTION OF THE EXEMPLARY EMBODIMENTS

Referring to FIGS. 1 through 4, there is illustrated an exemplary embodiment of a fluid delivery device 10 constructed in accordance with the present invention, which can be used for the delivery of fluids to a person or animal. The fluid delivery device 10 is provided with exemplary embodiments of new and improved low profile components 12, 14, 16 constructed in accordance with the present invention. The components 12, 14, 16 of the fluid delivery device 10 of the present invention have relatively low profiles (i.e., heights) so that the resulting fluid delivery device 10 also has a relatively low profile when attached to the skin of a patient. Among other features and benefits, the low profile fluid delivery device 10 is less discrete during use, can more easily fit under the clothing of a patient when attached to the patient's skin, and is less likely to be accidentally removed from the patient's skin. Moreover, the low profile components 12, 14, 16 of the fluid delivery device 10 allow the components to be vertically stacked without increasing the overall height of the device 10. Vertically stacking the components 12, 14, 16, in turn, lends the device 10 to mass production techniques so that devices constructed in accordance with the present invention can be made relatively cheaply and can be disposable in nature. In the exemplary embodiment of FIGS. 1 through 4, the low profile components include a reservoir 12 for holding fluid for infusion, a dispenser 14 for causing fluid to flow from the reservoir 12 during infusion, and a power supply 16, such as a battery or capacitor, supplying power to the dispenser 14.

Before the low profile components 12, 14, 16 are discussed in further detail, however, the fluid delivery device 10 will first be described to provide some background information. The types of liquids that can be delivered by the fluid delivery device 10 include, but are not limited to, insulin, antibiotics, nutritional fluids, total parenteral nutrition or TPN, analgesics, morphine, hormones or hormonal drugs, gene therapy drugs, anticoagulants, analgesics, cardiovascular medications, AZT or chemotherapeutics. The types of medical conditions that the fluid delivery device 10 might be used to treat include, but are not limited to, diabetes, cardiovascular disease, pain, chronic pain, cancer, AIDS, neurological diseases, Alzheimer's Disease, ALS, Hepatitis, Parkinson's Disease or spasticity. The volume of the reservoir 12 of the fluid delivery device 10 is chosen to best suit the therapeutic application of the fluid delivery device 10 impacted by such factors as available concentrations of medicinal fluids to be delivered, acceptable times between refills or disposal of the fluid delivery device 10, size constraints and other factors.

The dispenser 14 causes fluid from the reservoir 12 to flow to a transcutaneous access tool, such as a skin penetrating cannula (not shown). Although not viewable, the fluid delivery device 10 also includes a processor or electronic microcontroller (hereinafter referred to as the “local” processor) connected to the dispenser 14, and programmed to cause a flow of fluid to the transcutaneous access tool based on flow instructions from a separate, remote control device 1000, an example of which is shown in FIG. 1. A wireless receiver is connected to the local processor for receiving flow instructions from the remote control device 1000 and delivering the flow instructions to the local processor.

The device 10 includes an external housing 18 containing the reservoir 12, the dispenser 14, the power supply 16, the local processor, and the wireless receiver. The housing 18 of the fluid delivery device 10 is preferably free of user input components for providing flow instructions to the local processor, such as electromechanical switches or buttons on an outer surface of the housing 18, or interfaces otherwise accessible to a user to adjust the programmed flow rate through the local processor. The lack of user input components allows the size, complexity and costs of the device 10 to be substantially reduced so that the device 10 lends itself to being small and disposable in nature. Examples of such devices are disclosed in co-pending U.S. patent application Ser. No. 09/943,992, filed on Aug. 31, 2001 (Atty. Docket No. INSL-110), and entitled DEVICES, SYSTEMS AND METHODS FOR PATIENT INFUSION, which is assigned to the assignee of the present application and has previously been incorporated herein by reference.

In order to program, adjust the programming of, or otherwise communicate user inputs to the local processor, the fluid delivery device 10 includes the wireless communication element, or receiver, for receiving the user inputs from the separate, remote control device 1000 of FIG. 1. Signals can be sent via a communication element (not shown) of the remote control device 1000, which can include or be connected to an antenna 1300, shown in FIG. 1 as being external to the device 1000.

The remote control device 1000 has user input components, including an array of electromechanical switches, such as the membrane keypad 1200 shown. The remote control device 1000 also includes user output components, including a visual display, such as a liquid crystal display (LCD) 1100. Alternatively, the control device 1000 can be provided with a touch screen for both user input and output. Although not shown in FIG. 1, the remote control device 1000 has its own processor (hereinafter referred to as the “remote” processor) connected to the membrane keypad 1200 and the LCD 1100. The remote processor receives the user inputs from the membrane keypad 1200 and provides “flow” instructions for transmission to the fluid delivery device 10, and provides information to the LCD 1100. Since the remote control device 1000 also includes a visual display 1100, the fluid delivery device 10 can be void of an information screen, further reducing the size, complexity and costs of the device 10.

The device 10 preferably receives electronic communication from the remote control device 1000 using radio frequency or other wireless communication standards and protocols. In a preferred embodiment, the communication element of the device 10 is a two-way communication element, including a receiver and a transmitter, for allowing the fluid delivery device 10 to send information back to the remote control device 1000. In such an embodiment, the remote control device 1000 also includes an integral communication element comprising a receiver and a transmitter, for allowing the remote control device 1000 to receive the information sent by the fluid delivery device 10.

The local processor of the device 10 contains all the computer programs and electronic circuitry needed to allow a user to program the desired flow patterns and adjust the program as necessary. Such circuitry can include one or more microprocessors, digital and analog integrated circuits, resistors, capacitors, transistors and other semiconductors and other electronic components known to those skilled in the art. The local processor also includes programming, electronic circuitry and memory to properly activate the dispenser 14 at the needed time intervals.

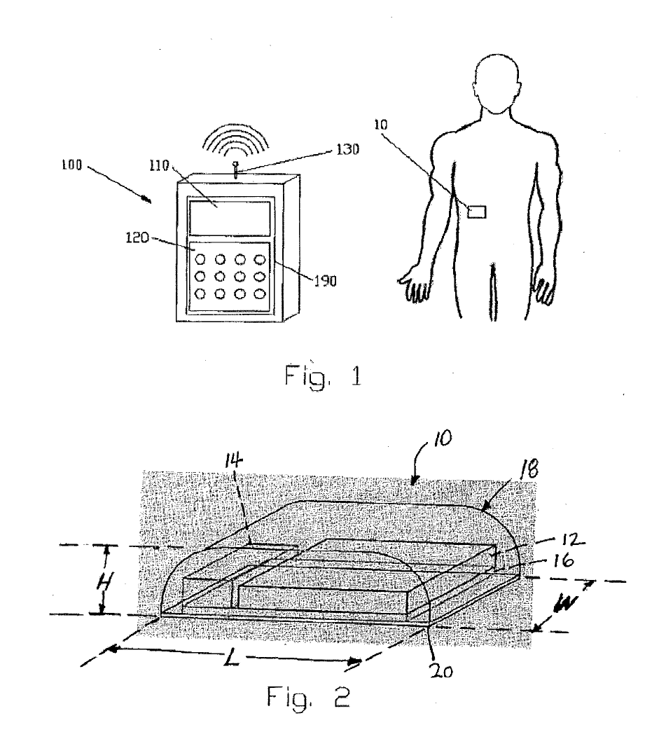

Referring now to FIGS. 2 through 4, in accordance with the present invention, at least two of the reservoir 12, the dispenser 14 and the power supply 16 are vertically stacked within the housing 18, and at least one of the dispenser 14 and the power supply 16 has a horizontal cross-sectional area that is greater than fifty percent of a cross-sectional area of the housing 18. In the exemplary embodiment of FIGS. 2 through 4, the reservoir 12 and the power supply 16 are vertically stacked within the housing 18, and the power supply 16 has a horizontal cross-sectional area that is greater than fifty percent of a cross-sectional area of the housing 18. Moreover, horizontal cross-sectional areas of the reservoir 12 and the power supply 16 overlap by at least fifty percent. (It should be noted that components shown in the drawings are not made to scale and are not necessarily shown in actual proportion to one another.)

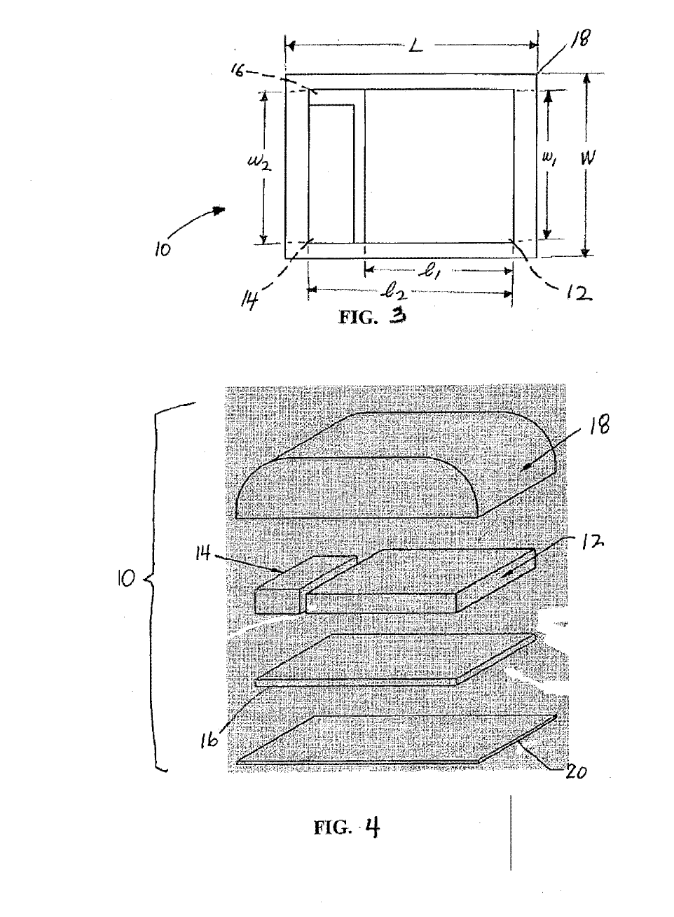

Referring to FIGS. 2 and 3, in the exemplary embodiment shown the cross-sectional area of the housing 18 is equal to a width “W” of the housing 18 multiplied by a length “L” of the housing 18. Referring to FIG. 3, in the exemplary embodiment shown the cross-sectional area of the reservoir 12 is equal to a width “w₁” of the reservoir 12 multiplied by a length “l₁” of the reservoir 12, and the cross-sectional area of the power supply 16 is equal to a width “w₂” of the power supply 16 multiplied by a length “l₂” of the power supply 16. If the power supply 16 comprises a battery, the flat geometry of the battery creates a large surface area to supply larger peak currents than similarly constructed batteries of smaller cross-sectional area. Larger peak currents are advantageous in various dispenser constructions such as those including dc motors, stepper motors and shaped memory components used as linear actuators.

Referring to FIG. 2, in the exemplary embodiment shown the housing 18 has a largest horizontal dimension equal to the length “L” of the housing 18, and the length “L” of the housing 18 is at least three (3) times greater than a largest vertical dimension of the housing. In the exemplary embodiment shown, the housing 18 has a largest vertical dimension equal to a height “H” of the housing 18. Moreover, the housing 18 has a smallest horizontal dimension equal to the width “W” of the housing 18, and the width “W” of the housing 18 is at least two (2) times the largest vertical dimension “H” of the housing 18. All of the features further ensure that the fluid delivery device 10 has a relatively low profile (i.e., height) above a surface 20 designed to contact the skin of a patient during use of the fluid delivery device 10 when attached to the skin of a patient. As shown best in FIGS. 2 and 4, the surface 20 for contacting the skin of a patient during use of the device 10 is part of a lower panel of the housing 18.

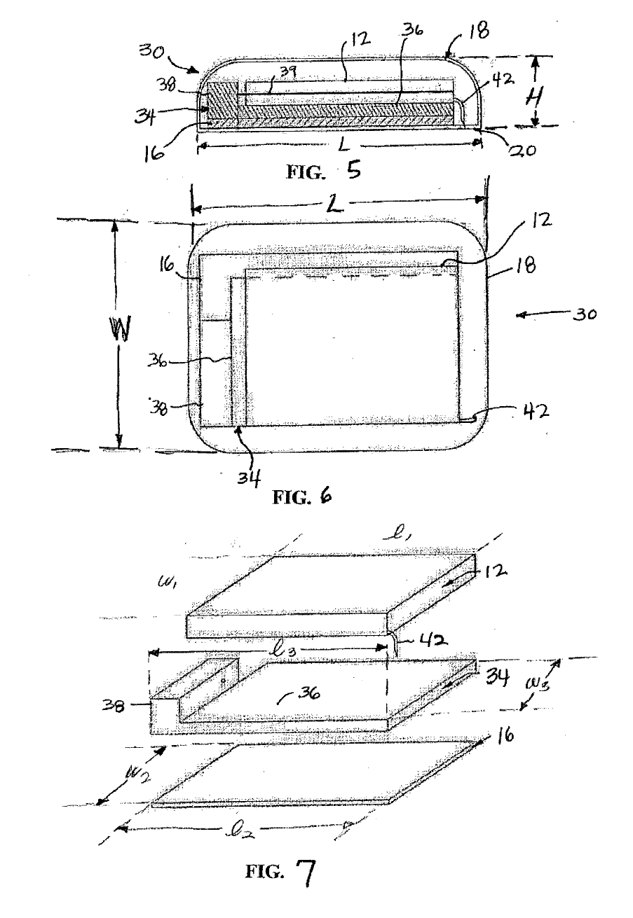

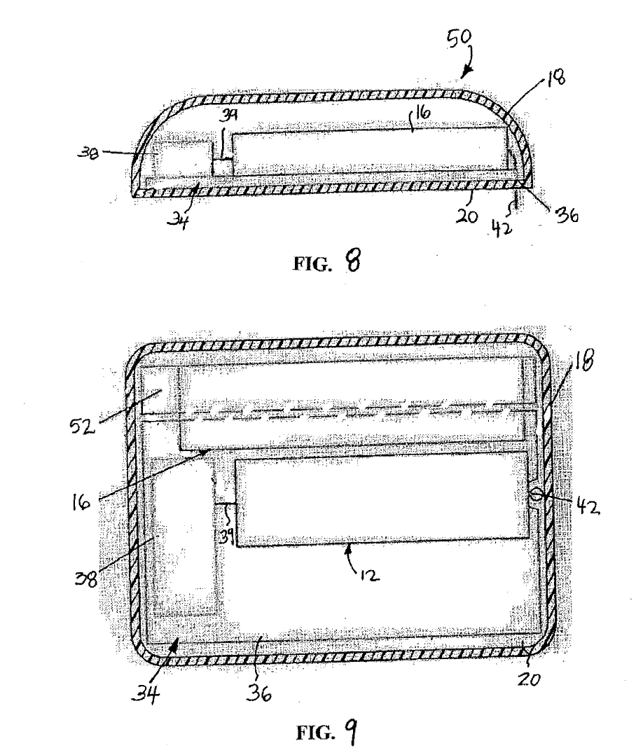

FIGS. 5 through 7 show another exemplary embodiment of a fluid delivery device 30 constructed in accordance with the present invention. The fluid delivery device 30 is generally similar to the fluid delivery device 10 of FIGS. 1 through 4 such that similar elements have the same reference numerals. However, in the fluid delivery device 30 of FIGS. 5 through 7, the reservoir 12, a dispenser 34 and the power supply 16 are vertically stacked within the housing 18 and the reservoir 12, the dispenser 34 and the power supply 16 each have a cross-sectional area that is greater than fifty percent of a cross-sectional area of the housing 18. In addition, horizontal cross-sectional areas of the reservoir 12, the dispenser 34 and the power supply 18 overlap by at least fifty percent. In the exemplary embodiment of the device 30, as shown in FIG. 7, the cross-sectional area of the dispenser 34 is equal to a width “w₃” of the dispenser 34 multiplied by a length “l₃” of the dispenser 34.

In the exemplary embodiment of FIGS. 5 through 7, the dispenser 34 includes a flat motor 36 operatively connected to the reservoir 12 such that operation of the motor 36 causes fluid from the reservoir 12 to flow to an exit port assembly 40 of the fluid delivery device 30. The motor 36 can comprise many possible embodiments for providing a motive force, such as a rotating drive shaft, for causing fluid to flow from the reservoir 12, as directed by the local processor of the device 30. For example, the motor 36 can comprise one or more of a DC motor or an AC motor, a spring-assisted motor, a stepper motor, a torque motor, a shaped memory element motor and a piezoelectric motor.

As shown, the motor 36 is vertically stacked with the reservoir 12 and a motive power converter 38 operatively connects the motor 36 to the reservoir 12. The motive power (e.g., torque) converter 38 is positioned on a side of the motor 36 and the reservoir 12 and is used to redirect motive power from the motor 36 to the reservoir 12. The motive power converter 38 can be adapted, for example, to re-direct torque from a rotating drive shaft of the motor 36 ninety degrees, or one-hundred and eighty degrees, into the reservoir 12, to thereby allow stacking of the motor 36 and the reservoir 12. In the exemplary embodiment of FIG. 5, a secondary drive shaft 39 extends from the motive converter 38 into the reservoir 12 and is adapted for causing fluid to flow from the reservoir 12. The secondary drive shaft 39 can be used, for example, to drive a piston in the reservoir 12 to thereby push fluid from the reservoir 12 upon operation of the motor 36.

The fluid delivery device 30 of FIGS. 5 through 7 also includes a transcutaneous access tool 42 for providing fluid communication between the reservoir 12 and a patient, through the bottom panel 20 of the housing 18. In the exemplary embodiment of FIGS. 5 through 7, the transcutaneous access tool comprises a soft cannula 42.

FIGS. 8 and 9 show another exemplary embodiment of a fluid delivery device 50 constructed in accordance with the present invention. The fluid delivery device 50 is generally similar to the fluid delivery device 30 of FIGS. 5 through 7 such that similar elements have the same reference numerals. However, in the fluid delivery device 50 of FIGS. 8 and 9, the reservoir 12 and the dispenser 32 are vertically stacked within the housing 18 and the dispenser 32 has a cross-sectional area that is greater than fifty percent of a cross-sectional area of the housing 18. In addition, horizontal cross-sectional areas of the reservoir 12 and the dispenser 32 overlap by at least fifty percent.

In the fluid delivery device 50 of FIGS. 8 and 9, the power supply 16 and a local processor 52 are vertically stacked within the housing 18, and horizontal cross-sectional areas of the power supply 16 and the processor 52 overlap by at least fifty percent.

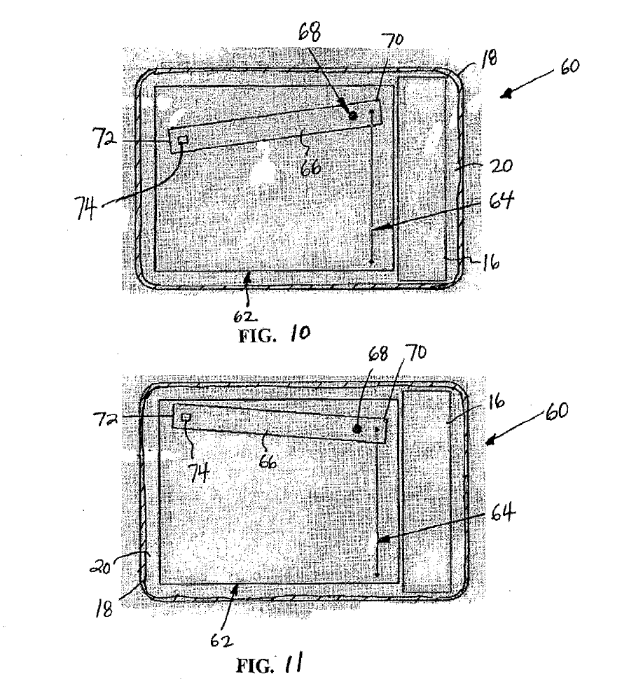

Referring to FIGS. 10 and 11, a portion of an exemplary embodiment of a fluid delivery device 60 constructed in accordance with the present invention is shown. The fluid delivery device 60 is generally similar to the fluid delivery devices of FIGS. 1 through 9 such that similar elements have the same reference numerals. The fluid delivery device 60 of FIGS. 10 and 11 includes a flat motor 62 vertically stacked within the housing 18 and the flat motor 62 has a cross-sectional area that is greater than fifty percent of a cross-sectional area of the housing 18.

The motor 62 includes a shape memory element 64 having a changeable length when at least one charge is applied to the shape memory element 64. The shape memory element 64 is made of a shape memory material such as a shaped memory alloy or shaped memory polymer. The application of an electrical current to a shape memory material results in molecular and crystalline restructuring of the shape memory material. If the shape memory material is in the shape of an elongated wire, for example, as the shape memory element 64 preferably is, this restructuring causes a decrease in length. Nitinol, a well-known alloy of nickel and titanium, is an example of such a so-called shape memory material and is preferred for use as the shape memory element 64. However, other types of shape memory material can be used.

The shape memory element 64 is operatively connected to the reservoir (not shown in FIGS. 10 and 11) such that the changeable length of the shape memory element 64 causes fluid to flow from the reservoir upon changing between an uncharged length and a charged length. The flat motor 62 also includes an elongated lever 66 mounted for pivotal movement about a pivot axis 68 located between opposing first and second ends 70, 72 of the lever 66. The lever 66 is arranged within the motor 62 so that the pivot axis 68 of the lever 66 extends perpendicular to the 20 base of the housing 18.

The shape memory element 64 is connected to the first end 70 of the lever 66 such that the changeable length of the shape memory element 64 causes pivotal movement of the lever 66 about the pivot axis 68, and the second end 72 of the lever 66 is operatively connected to the reservoir such that pivotal movement of the lever 66 about the pivot axis 68 causes fluid to flow from the reservoir. In the exemplary embodiment shown, the changeable length of the shape memory element decreasing from an uncharged length to a charged length causes pivotal movement of the lever about the pivot axis. Although not shown, the lever 66 is biased about the pivot axis 68, by a helical spring for example, such that the biased lever 66 returns to its original position (shown in FIG. 10) upon the charge being removed from the shape memory element 64.

In the exemplary embodiment of FIGS. 10 and 11, the second end 72 of the lever 66 is operatively connected to the reservoir at least in part through a finger 74 secured to the second end 72 of the lever 66. Upon successively applying a charge to and removing a charge from the shape memory element 64, the finger 74 is moved in a reciprocating manner. The finger 74 in turn can be coupled to a motion transfer mechanism (e.g., a ratchet mechanism, lead screw and plunger assembly) operatively connected to the reservoir of the device 60 such that reciprocating motion of the finger 74 causes fluid to flow from the reservoir.

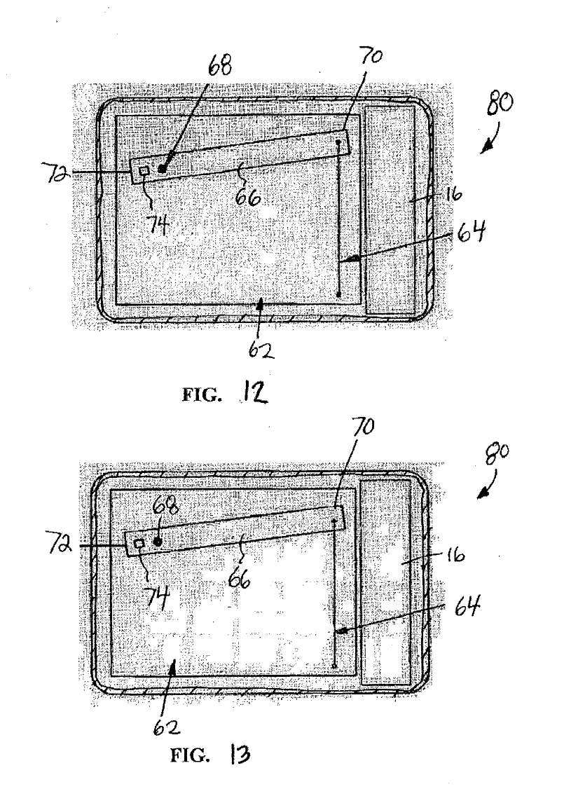

In the exemplary embodiment of FIGS. 10 and 11, the pivot axis 68 of the lever 66 is positioned closer to the first end 70 than the second end 72 of the lever 66. In this embodiment, the shaped memory element 64 produces a relatively large displacement of the second end 72 of the lever 66. FIGS. 12 and 13 show another exemplary embodiment of a fluid delivery device 80 constructed in accordance with the present invention. The fluid delivery device 80 is generally similar to the fluid delivery device 60 of FIGS. 10 and 11 such that similar elements have the same reference numerals. However, in the fluid delivery device 80 of FIGS. 12 and 13, the pivot axis 68 of the lever 66 is positioned closer to the second end 72 than the first end 70 of the lever 66. In this embodiment, the shaped memory element 64 produces a relatively small displacement of the second end 72 of the lever 66, but displaces the second end 72 with a relatively greater force. It should be noted that levers such as those described in FIGS. 10-11 and 12-13 can be advantageous in designs other than those using shaped memory elements, e.g., other actuators, such as linear actuators, magnetic actuators, solenoids, piezo crystal actuators, etc.

Referring to FIGS. 14 and 15, a portion of another exemplary embodiment of a fluid delivery device 90 constructed in accordance with the present invention is shown. The fluid delivery device 90 is generally similar to the fluid delivery devices of FIGS. 1 through 13 such that similar elements have the same reference numerals. The fluid delivery device 90 of FIGS. 14 and 15 includes a flat motor 92 vertically stacked within the housing 18 and the flat motor 92 has a cross-sectional area that is greater than fifty percent of a cross-sectional area of the housing 18. As shown in FIG. 15, the reservoir 12 of the device 90 is stacked on the flat motor 92, and the flat motor 92 is operatively connected to the reservoir 12 through a motive power (e.g., torque) converter 38 having a lead screw 39 extending into the reservoir 12. A plunger 37 is operatively mounted on the lead screw 39 so that the plunger 37 moves within the reservoir 12 upon rotation of the lead screw 39, to force fluid from the reservoir 12.

Referring to FIG. 14, the flat motor 92 includes a shape memory element 94, which changes shape upon the application of an electrical charge to the element 94. The shape memory element 94 is elongated and is anchored in place at a first end 96 and connected at a second end 98 to a member 100 that is reciprocally movable in opposing directions. The member 100 includes a finger 102 extending therefrom which interacts with the torque converter 38, shown in FIG. 15, so that reciprocating movement of the member 100 causes rotation of the lead screw 39 and movement of the plunger 37 within the reservoir 12.

The shape memory element 94 is adapted and arranged such that the member 100 is moved in a first direction upon an electric charge being applied to the shape memory element 94. The member 100 is biased in a second, opposite direction by a helical spring 104 so that the biased member 100 returns to its original position upon the charge being removed from the shape memory element 94. Successively applying electrical charges to the shape memory element 94, therefore, causes reciprocating movement of the member 100.

As shown in FIG. 14, the shape memory element 94 is elongated and circuitously wound through a plurality of posts 96. If desired, the posts 96 can be made of low friction material or can be rotatable in order to more easily allow movement of the shape memory element 94. Circuitously winding the shape memory element 94 through the posts 96 allows a longer shape memory element 94 to be provided without unduly enlarging the length or width of the flat motor 92, so that the shape memory element 94 can produce a relatively large displacement of the member 100 upon being charged.

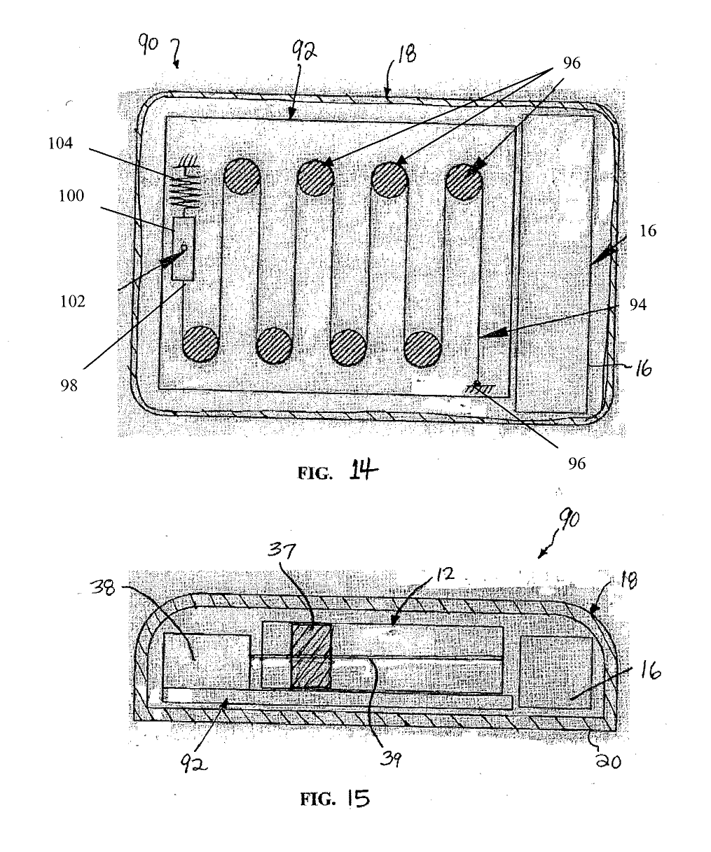

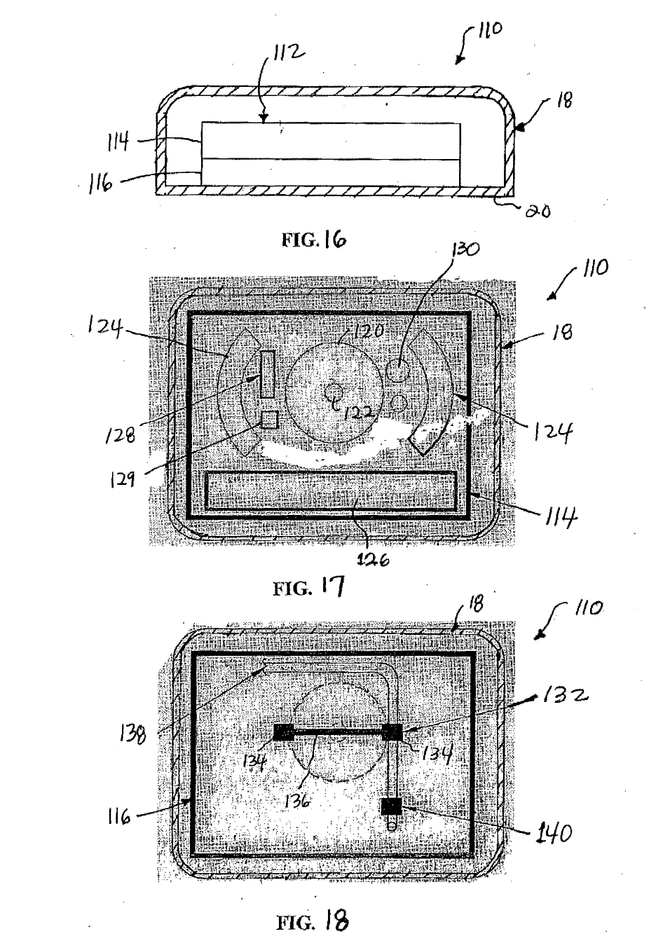

FIGS. 16, 17 and 18 show an additional exemplary embodiment of a fluid delivery device 110 constructed in accordance with the present invention. As shown best in FIG. 16, the fluid delivery device 110 includes a dispenser 112 having a flat motor 114 stacked over a motive power converter 116.

Referring to FIG. 17, the flat motor 114 includes, among other components, a rotor 120 secured to a drive shaft 122, and fixed magnets 124 arranged around the rotor 120. The components 120, 124 of the motor 114, however, are not provided in a separate, individual package, but are instead manufacture as an integrated portion of the fluid delivery device 110. The components 120, 124 of the motor 114 are preferably spaced apart by a largest distance greater than at least the largest vertical dimension of the housing. A power supply 126 provides power to the rotor 120, and miscellaneous electronics 128, 129, 130 (e.g., capacitors, inductors, semiconductors, etc.) are provide between the components 120, 124 of the flat motor 114. The relatively large separation of the components 120, 124 of the flat motor 114 allows for more specific DC motor designs. The flat motor 114 is particularly suitable for automated, mass manufacturing construction techniques, and eliminates motor packaging costs.

Other components can be provided between the motor components. For example, a cannula injection assembly (in whole or in part), housing support members, sensors (such as pressure sensors), portions of the flow path, etc., can all be provided between the motor components. The exploded design of the motor allows the most efficient use of space within the compact, low profile design of the fluid delivery device. The non-motor components placed between the motor components allows for more compact pump design. Components that do not interfere with the electromagnetic fields of the motor, e.g. plastic components, may be best suited for this interspersed design concept, but some passive electronic components may be compatible as well.

Referring to FIG. 18, the motive power converter 116 includes a rotatable roller assembly 132 including multiple rollers 134 connected to a central hub 136. Each roller 134 is positioned in contact with a portion of fluid transport tube 138 connected to a reservoir (not shown) of the fluid delivery device 10. The drive shaft 122 of the flat motor 114 of FIG. 17 is operatively connected to the central hub 136 such that the roller assembly 132 rotates upon operation of the flat motor 114. The rotating roller assembly 132, in turn, acts as a peristaltic pump and causes fluid to be drawn through the fluid transport tube 138. The motive power converter 116 also includes a check valve 140 controlling fluid flow through the fluid transport tube 138. The check valve 140 is itself controlled by local processor of the fluid delivery device 10, and acts to prevent inadvertent fluid flow.

Referring to FIGS. 19 and 20, a further exemplary embodiment of a fluid delivery device 150 constructed in accordance with the present invention is shown. The fluid delivery device 150 is generally similar to the fluid delivery devices of FIGS. 1 through 18 such that similar elements have the same reference numerals. The fluid delivery device 150 of FIGS. 19 and 20 includes a flat motor 152 vertically stacked within the housing 18 and the flat motor 152 has a cross-sectional area that is greater than fifty percent of a cross-sectional area of the housing 18. A rigid and rotatable backing plate 154 supports a reservoir 156, as also shown in FIG. 22, stacked on the flat motor 152. As directed by the local processor of the device 150, the motor 152 causes the backing plate 154 and the reservoir 156 to rotate above the motor 152 in order to cause fluid to flow from the reservoir 156 as described in greater detail below. The motor 152 can comprise many possible embodiments for providing a motive force, such as a DC motor or an AC motor, a spring-assisted motor, a stepper motor, a torque motor, a shaped memory element motor, or a piezoelectric motor.

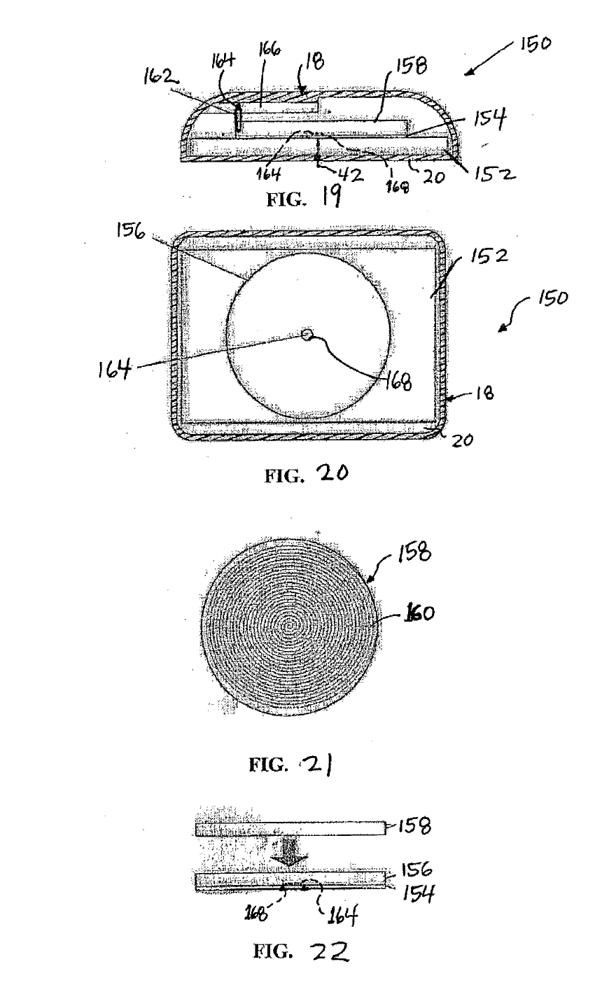

The reservoir 156 is flexible and a pin guide 158 defining a passageway 160 (shown best in FIG. 21) is received over the reservoir 156 (as shown in FIG. 22) such that the flexible reservoir 156 is sandwiched between the pin guide 158 and the backing plate 154 and fills the passageway 160 of the pin guide 158. The pin guide 158 and the reservoir 156 move with the backing plate 154, and a pin 162 extends into the passageway 160 of the pin guide 158 generally perpendicular to the backing plate 154 and is movable in a direction parallel to the backing plate 154, such that movement of the backing plate 154 causes the pin 162 to move along the passageway 160 of the pin guide 158, towards an outlet 164 of the reservoir 156, and successively collapse the reservoir 156 and force fluid through the outlet 164. The pin 162 is biased towards the backing plate 154 by a spring 164, shown in FIG. 19, and is movable along a channel 166 in a direction parallel with the backing plate 154.

In the exemplary embodiment shown in FIG. 21, the passageway 160 of the pin guide 158 begins at an outer circumference of the pin guide 158, ends in a center of the pin guide 158 and extends in a spiral path between the outer circumference and the center of the pin guide 158. The backing plate 154 has a central opening 168 aligned with the center of the pin guide 158, the outlet 164 of the reservoir 156 is aligned with the center of the pin guide 158, and a cannula or needle 42 extends from the outlet 164, through the central opening 168 of the backing plate 154 and through the base 20 of the housing 18 for insertion into a patient's skin.

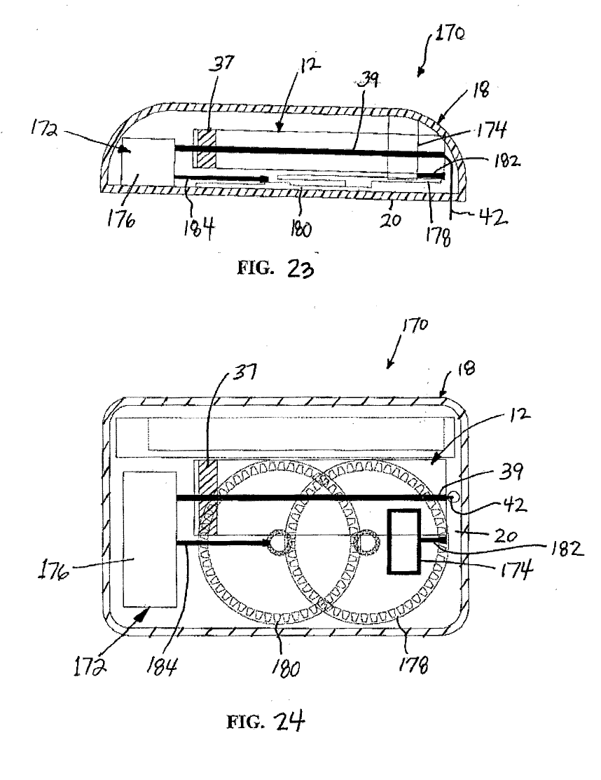

Referring to FIGS. 23 and 24, still another exemplary embodiment of a fluid delivery device 170 constructed in accordance with the present invention is shown. The fluid delivery device 170 is generally similar to the fluid delivery devices of FIGS. 1 through 22 such that similar elements have the same reference numerals. The fluid delivery device 170 of FIGS. 23 and 24 includes a flat dispenser 172 vertically stacked within the housing 18 and the flat dispenser 172 has a cross-sectional area that is greater than fifty percent of a cross-sectional area of the housing 18.

The dispenser 172 includes a motor 174 and a torque converter 176. As shown, the motor 174 is vertically stacked with a reservoir 12 and the torque converter 176 operatively connects the motor 174 to the reservoir 12. The torque converter 176 is positioned on a side of the motor 174 and the reservoir 12 and is used to redirect torque from the motor 174 to the reservoir 12. The torque converter 176 is adapted to re-direct torque from a rotating drive shaft of the motor 174 one-hundred and eighty degrees into the reservoir 12, to thereby allow stacking of the motor 174 and the reservoir 12. A driven shaft, or lead screw 39 extends from the torque converter 176 into the reservoir 12 and is adapted drive a piston 37 in the reservoir 12, which has a side wall extending towards an outlet, to thereby push fluid from the reservoir 12 and through a transcutaneous access tool 42 upon operation of the motor 174.

The dispenser 172 includes at least one motor gear 178 having an axis of rotation extending perpendicular to a base 20 of the housing 18 of the device, and the motor gear has a diameter greater than a largest vertical dimension of the housing 18. The motor gear 178 also has an area defined by a diameter of the gear that is greater than about fifty percent of the cross-sectional area of the housing 18. The relatively large diameter of the motor gear 178 allows significant gear reduction, which is desirable in producing a small step size or advancement of the plunger 37 within the reservoir 12, as well as high torque. The relatively large diameter of the motor gear 178 and the arrangement of the motor gear 178 also allows the dispenser 172 to have a relatively low profile.

In the exemplary embodiment of FIGS. 23 and 24, the dispenser 172 includes a first motor gear 178 and a second motor gear 180. The dispenser 172 also includes a first drive shaft 182 extending from the motor 174 and a second drive shaft 184 extending from the torque converter 176. The first drive shaft 182 engages radially outer teeth of the first motor gear 178, radially inner teeth of the first motor gear 178 engage radially outer teeth of the second motor gear 180, and radially inner teeth of the second motor gear 180 engages the second drive shaft 184, so that rotation of the first drive shaft 182 causes rotation of the second drive shaft 184, and rotation of the lead screw 39 within the reservoir 12.

Although not shown, the torque converter 176 can contain a gear train, such as a drive gear operatively connected to the second drive shaft 184, a driven gear operatively connected to the drive gear such that rotation of the drive gear causes rotation of the driven gear, and the lead screw 39 connected to the driven gear for rotation with the driven gear. Intermediate gears can also be provided between the drive gear and the driven gear.

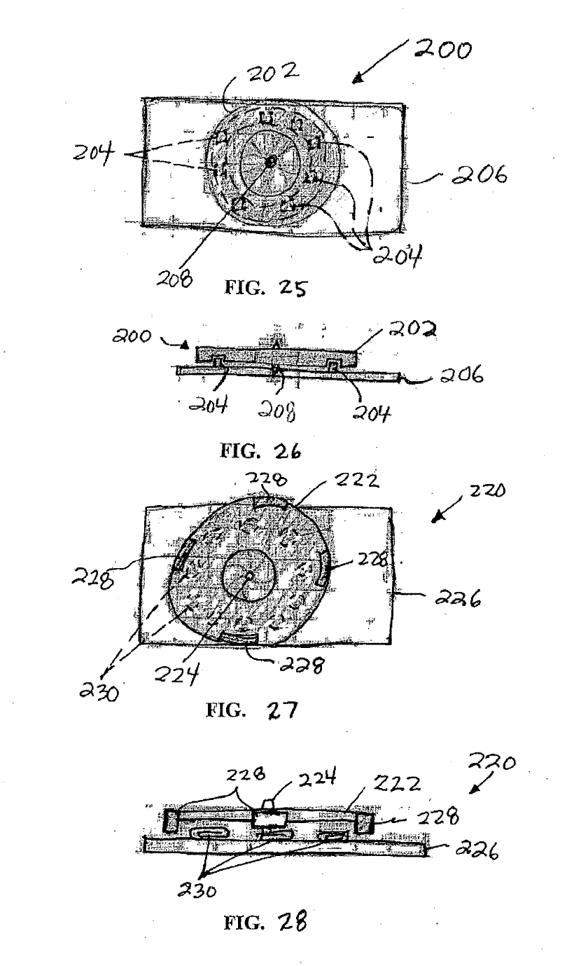

Referring to FIGS. 25 and 26, an exemplary embodiment of an electric motor 200 constructed in accordance with the present invention is shown. The motor 200 is for use as part of a dispenser of a low profile fluid delivery device, such as the fluid delivery devices shown in FIGS. 1 through 24. The motor 200 includes parts, such as a stator/rotor 202 and magnets 204, that are manufactured on a printed circuit board 206. As shown in FIGS. 25 and 26, the stator/rotor 202 is rotatably mounted on a shaft 208 attached to the printed circuit board 206, and the magnets 204 are fixedly attached to the printed circuit board 206 beneath the stator/rotor 202. Electrifying the fixed magnets 204, therefore, causes the stator/rotor 202 to rotate about the shaft 208.

Although not shown, other electronic components of the motor 200 and electronic components of the fluid delivery device utilizing the motor can be mounted on the printed circuit board 206. Manufacturing the motor 200 as part of the printed circuit board 206 is conducive to providing low profile components for a fluid delivery device and allows a more efficient use of available space within the device. The printed circuit board motor is also a relatively low-cost, highly manufacturable design.

FIGS. 27 and 28 show another exemplary embodiment of an electric motor 220 constructed in accordance with the present invention. The motor 220 is similar to the motor 200 of FIGS. 25 and 26. However, the motor 220 of FIGS. 27 and 28 includes a rotor 222 that is rotatably mounted on a shaft 224 attached to a printed circuit board 226, and magnets 228 are secured to the rotor 222, and the motor 220 further includes stator segments 230 that are fixedly attached on or in the printed circuit board 226. Electrifying the fixed stator segments 230, therefore, causes the rotor 222 and the magnets 228 to rotate about the shaft 224.

As illustrated by the above described exemplary embodiments, the present invention generally provides new and improved low profile components for a device for delivering fluid, such as insulin for example, to a patient. It should be understood that the embodiments described herein are merely exemplary and that a person skilled in the art may make variations and modifications to the embodiments described without departing from the spirit and scope of the present invention. For example, other low profile components can include a heater or a cooling unit (e.g. a heat sink) to regulate the temperature of fluid within the reservoir, an antenna assembly (passive or active), a sensor assembly (e.g. a physiologic sensor such as a glucose sensor or an internal sensor such as a pressure sensor), a cannula injection assembly (laying flat to get large “sweeps” of injection mechanism), and dispenser in the form of an accumulator and a valve assembly, and a skin attachment mechanism. If it is desired to provide the device with user interface components, the user interface components can also be provide with a low profile. All such equivalent variations and modifications are intended to be included within the scope of this invention as defined by the appended claims.

What is claimed is:

1. A device for delivering fluid to a patient, comprising:
a reservoir;
a dispenser for causing fluid to flow from the reservoir;
a local processor connected to the dispenser and programmed to cause a flow of fluid from the reservoir based solely on flow instructions from a separate, remote control device;
a power supply connected to the local processor; and
a housing containing the reservoir, the dispenser, the local processor, the power supply and the wireless receiver;
wherein at least two of the reservoir, the dispenser and the power supply are vertically stacked within the housing and at least one of the dispenser and the power supply has a horizontal cross-sectional area that is greater than fifty percent of a cross-sectional area of the housing.

2. A device according to claim 1, further comprising a wireless receiver connected to the local processor for receiving the flow instructions from a separate, remote control device and delivering the flow instructions to the local processor.

3. A device according to claim 1, wherein the housing is free of user input components for providing flow instructions to the local processor.

4. A device according to claim 1, further comprising a transmitter connected to the local processor for transmitting information from the local processor to a separate, remote control device.

5. A device according to claim 4, wherein the housing is free of user output components for providing information from the local processor.

6. A device according to claim 1, further comprising a transcutaneous patient access tool connected to the reservoir.

7. A device according to claim 1, wherein the reservoir contains a therapeutic fluid.

8. A device according to claim 1, wherein the reservoir contains insulin.

9. A system including a fluid delivery device according to claim 2, and further comprising a remote control device separate from the fluid delivery device and including:
a remote processor;
user interface components connected to the remote processor for allowing a user to provide flow instructions to the remote processor; and
a transmitter connected to the remote processor for transmitting the flow instructions to the receiver of the fluid delivery device.

10. A system according to claim 9, wherein:
the fluid delivery device includes a transmitter connected to the local processor for transmitting information from the local processor; and
the remote control device includes a receiver connected to the remote processor for receiving the information from the transmitter of the fluid delivery device.

11. A system according to claim 10, wherein the housing of the fluid delivery device is free of user output components.

12. A device according to claim 1, wherein the housing has a largest horizontal dimension equal to at least three times a largest vertical dimension of the housing.

13. A device according to claim 1, wherein the housing has a smallest horizontal dimension equal to at least two times a largest vertical dimension of the housing.

14. A device according to claim 1, wherein the reservoir and the power supply are vertically stacked within the housing and the power supply has a horizontal cross-sectional area that is greater than fifty percent of a horizontal cross-sectional area of the housing.

15. A device according to claim 14, wherein horizontal cross-sectional areas of the reservoir and the power supply overlap by at least fifty percent.

16. A device according to claim 1, wherein the reservoir, the dispenser and the power supply are vertically stacked within the housing and the reservoir, the dispenser and the power supply each have a cross-sectional area that is greater than fifty percent of a cross-sectional area of the housing.

17. A device according to claim 16, wherein horizontal cross-sectional areas of the reservoir, the dispenser and the power supply overlap by at least fifty percent.

18. A device according to claim 1, wherein the reservoir and the dispenser are vertically stacked within the housing and the dispenser has a cross-sectional area that is greater than fifty percent of a cross-sectional area of the housing.

19. A device according to claim 18, wherein horizontal cross-sectional areas of the reservoir and the dispenser overlap by at least fifty percent.

20. A device according to claim 1, wherein the dispenser comprises a motor operatively connected to the reservoir such that operation of the motor causes fluid from the reservoir to flow to the exit port assembly.

21. A device according to claim 20, wherein the motor is vertically stacked with the reservoir and a torque converter, wherein the torque converter operatively connects the motor to the reservoir and the torque converter is not vertically stacked with the reservoir.

22. A device according to claim 1, wherein the dispenser comprises a motor having a movable drive member and a torque converter operatively connecting the moveable drive member of the motor to the reservoir such that movement of the drive member causes fluid to flow from the reservoir.

23. A device according to claim 22, wherein:
the movable drive member of the motor comprises a rotatable drive shaft; and
the torque converter comprises,
a drive gear secured to the drive shaft for rotation with the drive shaft,
a driven gear operatively connected to the drive gear such that rotation of the drive gear causes rotation of the driven gear, and
a driven shaft connected to the driven gear for rotation with the driven gear, wherein the driven shaft is operatively connected to the reservoir so that rotation of the driven shaft causes fluid to flow from the reservoir.

24. A device according to claim 23, wherein the reservoir includes a side wall extending towards an outlet, the driven shaft of the torque converter is received at least partly in the reservoir and longitudinally extends towards the outlet, and a plunger is secured to the driven shaft and has an outer periphery linearly slideable along the side wall of the reservoir, and wherein the plunger is operatively connected to the driven shaft such that rotation of the driven shaft causes movement of the plunger towards the outlet.

25. A device according to claim 24, wherein the motor includes at least one motor gear having an axis of rotation extending perpendicular to a base of the housing of the device and wherein the motor gear has a diameter greater than a largest vertical dimension of the housing.

26. A device according to claim 24, wherein the motor includes at least one motor gear having an axis of rotation extending perpendicular to a base of the housing of the device and wherein the motor gear has an area defined by a diameter of the gear that is greater than about fifty percent of the cross-sectional area of the housing.

27. A device according to claim 24, wherein the drive shaft of the motor comprises a first drive shaft and a second drive shaft and the motor includes at least two motor gears operatively connecting the first and the second drive shafts so that rotation of the first drive shaft causes rotation of the second drive shaft, and wherein the motors gears each have an axis of rotation extending perpendicular to a base of the housing of the device.

28. A device according to claim 27, wherein the motor gears each have a diameter greater than a largest vertical dimension of the housing.

29. A device according to claim 27, wherein the motor gears each have an area defined by a diameter of the gear that is greater than about fifty percent of the horizontal cross-sectional area of the housing.

30. A device according to claim 27, wherein the first drive shaft engages radially outer teeth of a first of the motor gears, radially inner teeth of the first motor gear engage radially outer teeth of a second of the motor gears, and radially inner teeth of the second motor gear engages the second drive shaft.

31. A device according to claim 1, wherein the dispenser comprises an shape memory element having a changeable length when at least one charge is applied to the shape memory element, wherein the shape memory element is operatively connected to the reservoir such that the changeable length of the shape memory element causes fluid to flow from the reservoir upon changing between an uncharged length and a charged length.

32. A device according to claim 31, wherein the dispenser further comprises an elongated lever mounted for pivotal movement about a pivot axis located between opposing first and second ends of the lever, and wherein the shape memory element is connected to the first end of the lever such that the changeable length of the shape memory element causes pivotal movement of the lever about the pivot axis and the second end of the lever is operatively connected to the reservoir such that pivotal movement of the lever about the pivot axis causes fluid to flow from the reservoir.

33. A device according to claim 32, wherein the pivot axis of the lever extends perpendicular to a base of the housing.

34. A device according to claim 32, wherein the pivot axis of the lever is positioned closer to the first end than the second end of the lever.

35. A device according to claim 32, wherein the pivot axis of the lever is positioned closer to the second end than the first end of the lever.

36. A device according to claim 32, wherein the changeable length of the shape memory element decreasing from an uncharged length to a charged length causes pivotal movement of the lever about the pivot axis.

37. A device according to claim 32, wherein the lever is biased about the pivot axis by a spring.

38. A device according to claim 31, wherein the shape memory element is elongated and is circuitously wound through a plurality of posts.

39. A device according to claim 38, wherein the posts are made of low friction material.

40. A device according to claim 38, wherein the posts are rotatable.

41. A device according to claim 38, wherein a first end of the shape memory element is fixed and a second end of the shape memory element is secured to a finger movable in a reciprocating manner.

42. A device according to claim 41, wherein the dispenser further includes a spring biasing the finger away from the shape memory element.

43. A device according to claim 1, wherein the dispenser comprises a rotatable roller assembly including multiple rollers connected to a central hub, wherein each roller is positioned in contact with a portion of fluid transport tube connected to reservoir.

44. A device according to claim 43, wherein the dispenser further comprises a check valve controlling fluid flow through the fluid transport tube.

45. A device according to claim 43, wherein the dispenser further comprises a motor operatively connected to the roller assembly.

46. A device according to claim 1, further comprising:
a movable rigid backing plate supporting the reservoir and wherein the reservoir is flexible and has an outlet;
a pin guide defining a passageway received over the reservoir such that the flexible reservoir is sandwiched between the pin guide and the backing plate and fills the passageway of the pin guide, and wherein the pin guide and the reservoir move with the backing plate; and
a pin extending into the passageway of the pin guide generally perpendicular to the backing plate and movable in a direction parallel to the backing plate such that movement of the backing plate causes the pin to move along the passageway of the pin guide and successively collapse the reservoir and force fluid through the outlet of the reservoir.

47. A device according to claim 46, wherein the movable backing plate rotates.

48. A device according to claim 47, wherein the passageway of the pin guide begins at an outer circumference of the pin guide, ends in a center of the pin guide and is spiral.

49. A device according to claim 48, wherein the backing plate has a central opening aligned with the center of the pin guide, the outlet of the reservoir is aligned with the center of the pin guide, and a tube extends from the outlet through the central opening of the backing plate.

50. A device according to claim 46, wherein the pin is biased towards the backing plate by a spring.

51. A device according to claim 46, wherein the dispenser includes a motor for moving the backing plate.

52. A device according to claim 1, wherein the dispenser comprises an electric motor including parts that are spaced apart by a largest distance greater than at least the largest vertical dimension of the housing.

53. A device according to claim 52, wherein the parts of the motor include a rotor and magnets.

54. A device according to claim 53, wherein the rotor is rotatably mounted on a shaft attached to a printed circuit board and the magnets are attached to the printed circuit board.

55. A device according to claim 53, wherein the rotor is rotatably mounted on a shaft attached to a printed circuit board and the magnets are secured to the rotor, and the motor further includes stators attached to the printed circuit board.

56. A device according to claim 52, wherein the largest distance the parts of the electric motor are spaced apart is greater than at least a largest horizontal dimension of the housing.

57. A device according to claim 1, wherein the dispenser includes a motor and a torque converter operatively connecting the motor to the reservoir such that operation of the motor causes fluid to flow from the reservoir.

58. A device according to claim 57, wherein the torque converter redirects torque from the motor by at least ninety degrees.

59. A device according to claim 57, wherein the torque converter redirects torque from the motor by at least one hundred and eighty degrees.

60. A device according to claim 1, wherein the dispenser includes a motor operatively connected to the reservoir such that operation of the motor causes fluid to flow from the reservoir.

61. A device according to claim 60, wherein the motor comprises a DC motor.

62. A device according to claim 60, wherein the motor comprises a stepper motor.

63. A device according to claim 60, wherein the motor comprises a torque motor.

64. A device according to claim 60, wherein the motor comprises a piezoelectric motor.

65. A device according to claim 60, wherein the motor comprises a shaped memory element.

66. A device according to claim 1, wherein the power supply comprises a battery.

67. A device according to claim 1, further comprising a connector connected to an outlet of the reservoir and connectable to a transcutaneous access device.

68. A device according to claim 1, further comprising a needle connected to an outlet of the reservoir.

69. A device according to claim 68, further comprising a mechanism for deploying the needle out of the housing of the device and into skin of a patient.

70. A device according to claim 2, wherein the receiver utilizes radio frequency signals.

71. A device according to claim 1, wherein the dispenser comprises at least one gear having an axis of rotation extending perpendicular to a base of the housing of the device and wherein the gear has a diameter greater than a largest vertical dimension of the housing.

72. A device according to claim 1, wherein the dispenser comprises a first drive shaft and a second drive shaft and at least two gears operatively connecting the first and the second drive shafts so that rotation of the first drive shaft causes rotation of the second drive shaft, and wherein the gears each have an axis of rotation extending perpendicular to a base of the housing of the device.

73. A device according to claim 1, wherein the dispenser comprises a first drive shaft and a second drive shaft and at least two gears operatively connecting the first and the second drive shafts so that rotation of the first drive shaft causes rotation of the second drive shaft, and wherein the gears each have a diameter greater than a largest vertical dimension of the housing.

74. A device according to claim 1, wherein the dispenser comprises a first drive shaft and a second drive shaft and at least two gears operatively connecting the first and the second drive shafts so that rotation of the first drive shaft causes rotation of the second drive shaft, and wherein the gears each have an area defined by a diameter of the gear that is greater than about fifty percent of the horizontal cross-sectional area of the housing.

75. A device according to claim 1, wherein the dispenser comprises an actuator operatively connected to the reservoir such that actuator causes fluid to flow from the reservoir upon being actuated.

76. A device according to claim 75, wherein the dispenser further comprises an elongated lever mounted for pivotal movement about a pivot axis located between opposing first and second ends of the lever, and wherein the actuator is connected to the first end of the lever such that the actuator causes pivotal movement of the lever about the pivot axis upon being actuated and the second end of the lever is operatively connected to the reservoir such that pivotal movement of the lever about the pivot axis causes fluid to flow from the reservoir.

77. A device according to claim 76, wherein the pivot axis of the lever extends perpendicular to a base of the housing.

78. A device according to claim 76, wherein the pivot axis of the lever is positioned closer to the first end than the second end of the lever.

79. A device according to claim 76, wherein the pivot axis of the lever is positioned closer to the second end than the first end of the lever.

80. A device according to claim 76, wherein the lever is biased about the pivot axis by a spring.
